(19) United States
(12) Patent Application Publication
(10) Pub. No.: US 2022/0339235 A1
(43) Pub. Date: Oct. 27, 2022

(54) TRADITIONAL CHINESE MEDICINE COMPOUND COMPOSITION WITH EFFECT OF IMPROVING MALE SEXUAL FUNCTION AND APPLICATION THEREOF

(71) Applicants: Chenland Nutritionals Inc., Irvine, CA (US); QINGDAO CHENLAND BIOLOGICAL TECHNOLOGY CO., LTD., Qingdao (CN)

(72) Inventors: Shengcan Zou, Qingdao (CN); Jie Wang, Qingdao (CN); Xin Li, Qingdao (CN); Li Li, Qingdao (CN); Shanglong Wang, Qingdao (CN); Yuanyuan Wang, Qingdao (CN); Xuena Song, Qingdao (CN)

(73) Assignees: Chenland Nutritionals Inc., Irvine, CA (US); QINGDAO CHENLAND BIOLOGICAL TECHNOLOGY CO., LTD., Qingdao (CN)

(21) Appl. No.: 17/859,028

(22) Filed: Jul. 7, 2022

(30) Foreign Application Priority Data
Dec. 27, 2021 (CN) 202111617184.2

(51) Int. Cl. A61K 36/88; A23L 33/105; A61K 36/746; A61K 36/71; A61K 36/64; A61K 36/815; A61K 36/43; A61P 15/08

(52) U.S. Cl. CPC A61K 36/88; A23L 33/105; A61K 36/746; A61K 36/71; A61K 36/64; A61K 36/815; A61K 36/43; A61P 15/08; A23V 2002/00

(57) ABSTRACT

A traditional Chinese medicine compound composition with effect of improving male sexual function and its application are disclosed. The disclosure prepares the traditional Chinese medicine compound composition by compounding Semen Allii Tuberosi with Radix Morindae Officinalis, Epimedii Folium, Cistanche deserticola Ma and Semen Cuscutae. The traditional Chinese medicine compound composition has a good effect on improving the sexual function of kidney Yang deficiency model mice. While improving sexual function, it can improve the abundance of spermatogenic cells in testicular seminiferous tubules, ensure that spermatogenic cells can continuously produce new sperm, and improve the number and morphology of sperm in the lumen. Moreover, the disclosed traditional Chinese medicine compound composition can be used as a raw material of dietary supplement or health food for the male population with low sexual function and easy fatigue. Therefore, the disclosed traditional Chinese medicine compound composition is suitable for market promotion and application.

### CROSS REFERENCE TO RELATED APPLICATION

This patent application claims the benefit and priority of Chinese Patent Application No. 202111617184.2 filed on Dec. 27,2021, the disclosure of which is incorporated by reference herein in its entirety as part of the present application.

### TECHNICAL FIELD

The present disclosure relates to the technical field of natural medicines, in particular to a formula of traditional Chinese medicine compound composition and its application, and more specifically, to a traditional Chinese medicine compound composition with effect of improving male sexual function and its application.

### BACKGROUND ART

Sex is a natural physiological and psychological phenomenon of human beings, and sexual function is the basis for human reproduction. Normal male sexual function is the premise for men to have sex and the guarantee of marital happiness. The studies found that with the acceleration of social rhythm and the increase of life pressure, the number of patients with sexual dysfunction has greatly increased due to the tense lifestyle, and the average age of patients is becoming younger and younger, threatening people's psychological health, physical health and happiness index.

The main factors that reduce the probability of pregnancy in women are usually congenital, immune, iatrogenic or endocrine reasons, while male hypospermia, sexual dysfunction and ejaculation dysfunction further lead to women being unable to conceive in many cases. The sexual function of normal men is a complex and complete process, including sexual desire, penis erection, orgasm and ejaculation, and these links affect each other. Problems with any one of them will affect the normal sexual life of men, that is, sexual dysfunction.

At present, although there are many ways to treat sexual dysfunction and significant breakthroughs have been made in the treatment of erectile dysfunction (ED) in recent years, many drugs still have many potential adverse reactions, which can not fundamentally solve the occurrence of the disease. At the same time, due to the high cost, a considerable number of patients can not afford it. Traditional Chinese medicine has a long history and rich experience in the treatment of this disease. The methods are simple, painless, with little toxic and side effects, low price, and easy for patients to accept.

Animal experiments or clinical trial studies have shown that oral western medicine has certain limitations and side effects, even serious adverse reactions. For example, Weige, which we are all familiar with, does have the function of aphrodisiac, but it may pose a serious threat to the life safety of patients with hypertension and angina pectoris. Another example is Viagra (Sildenafil Citrate), which is currently the first-line oral drug for the treatment of ED, also causes adverse reactions to varying degrees, such as headache, dizziness, nasal congestion, priapism, etc. In particular, due to drug synergy, there is incompatibility between Viagra and nitrous acid drugs, and long-term use of sildenafil will lead to penis fibrosis and other serious consequences.

Therefore, it is an urgent problem for those skilled in the art to develop a traditional Chinese medicine compound composition that can be used as a dietary supplement or health food raw material to improve male sexual function.

### SUMMARY

In view of the above, an object of the present disclosure is to aim at the problems existing in the prior art and to provide a traditional Chinese medicine compound composition with effect of improving male sexual function and its application in the preparation of health food.

It should be noted that all the raw materials of the disclosure can be used as health food raw materials, with a long history of eating, safe and non-toxic. While improving male sexual function, it can improve the abundance of spermatogenic cells in testicular seminiferous tubules and ensure that spermatogenic cells can continuously produce new sperm. It can not only improve the quantity and morphology of sperm in the lumen and improve the quality of sperm, but also avoid the problem of large side effects of traditional western medicine. And the product adopts modern extraction technology to extract and refine the components of traditional Chinese medicine, avoiding the problems of unstable quality of traditional Chinese medicine and inconvenient use of traditional decoction.

In order to achieve the above purpose, technical solutions of the present disclosure are specifically described as follows.

A traditional Chinese medicine compound composition with effect of improving male sexual function is provided. The traditional Chinese medicine compound composition is prepared by mixing following raw materials after separate extraction, and the raw materials include:

1˜10 parts by weight of Semen Allii Tuberosi;

1˜10 parts by weight of Radix Morindae Officinalis;

1˜10 parts by weight of Epimedii Folium;

or

1˜10 parts by weight of Semen Allii Tuberosi;

1˜10 parts by weight of Radix Morindae Officinalis;

1˜10 parts by weight of Cistanche desert/cola Ma;

or

1˜10 parts by weight of Semen Allii Tuberosi;

1˜10 parts by weight of Lycium barbarum; p 1˜10 parts by weight of Semen Cuscutae.

The disclosure provides a traditional Chinese medicine compound composition with effect of improving male sexual function, which is prepared by combining Semen Allii Tuberosi, Radix Morindae Officinalis, Epimedii Folium, Cistanche deserticola Ma, Lycium barbarum and Semen Cuscutae through separate extraction and mixing. Through the pharmacodynamic test, it was verified that it had a good effect on improving the sexual function of kidney Yang deficiency model mice. While improving their sexual function, they can improve the abundance of spermatogenic cells in testicular seminiferous tubules, ensure that spermatogenic cells can continuously produce new sperm, and improve the number and morphology of sperm in the lumen. The traditional Chinese medicine compound composition is different from the hormone aphrodisiac products on the market, has no side effects, is safe and easy to absorb, and is suitable for popularization and application.

It should be noted that the amount of the traditional Chinese medicine extracts added to the compound composition disclosed and protected by the present disclosure is reasonable, ranging from 2 to 5 kinds. Through the guidance of network pharmacology and traditional Chinese medicine theory, the types of traditional Chinese medicine are screened, the main active substances are determined, and it is verified through animal efficacy test, so that the traditional Chinese medicine compound composition with effect of improving male sexual function can achieve the purpose of quick effect, significant effect, small daily dosage and low cost.

In addition, the traditional Chinese medicine materials used in the traditional Chinese medicine extracts of the disclosure are scientifically selected according to scientific basis and organically combined according to the compatibility of traditional Chinese medicines rather than simple superimposing of the effect of each single Chinese medicine. The specific functions of various abovementioned traditional Chinese medicine materials are as follows.

Epimedii Folium: acrid, sweet and warm, belonging to liver meridian and kidney meridian. It can tonify kidney Yang, strengthen muscles and bones, and dispel wind dampness. It is used for kidney Yang deficiency, impotence and spermatorrhea, flaccid muscles and bones, rheumatism and arthralgia, numbness and contracture.

Semen Allii Tuberosi: acrid, sweet and warm, belonging to liver meridian and kidney meridian. It can mildly tonify liver and kidney, strengthen Yang and secure essence. It is used for liver and kidney deficiency, waist and knee pain, impotence, spermatorrhea, enuresis, frequent urination and leucorrhea.

Radix Morindae Officinalis: sweet, acrid, mild, belonging to kidney meridian and liver meridian. It can tonify kidney Yang, strengthen muscles and bones, and dispel wind dampness. It is used for impotence and spermatorrhea, uterine cold infertility, irregular menstruation, abdominal cold pain, rheumatism and arthralgia, and flaccid muscles and bones.

Cistanche deserticola Ma: acrid, salty, warm, belonging to kidney meridian and large intestine meridian. It can tonify kidney Yang, benefit blood essence, moisten intestines and relieve constipation. It is used for kidney Yang deficiency, blood essence deficiency, impotence and infertility, waist and knee soreness, muscle and bone weakness, intestinal dryness and constipation.

Lycium barbarum: sweet, normal, belonging to liver meridian and kidney meridian. It can nourish liver and kidney, benefit essence and brighten eyes. It is used for asthenia and essence deficiency, waist and knees soreness, dizziness and tinnitus, impotence and spermatorrhea, internal heat and thirst, blood deficiency and withered-yellow, and blurred vision.

Semen Cuscutae: acrid, sweet and normal, belonging to liver meridian, kidney meridian and spleen meridian. It can tonify liver and kidney, secure essence and shrink urine, prevent abortion, brighten eyes, and stop diarrhea. It is used for dispersing wind and removing freckle, liver and kidney deficiency, waist and knee soreness, impotence and spermatorrhea, enuresis, frequent urination, kidney deficiency, fetal leakage, fetal movement, dizzy eyes, tinnitus, spleen and kidney deficiency, and diarrhea.

In addition, modern pharmacology research shows that Semen Cuscutae can significantly improve sperm capillary penetration value, movement speed and sperm activity index, and increase sperm motility. Moreover, Semen Cuscutae extract can protect the integrity of sperm membrane and maintain the integrity of acrosome. These protective effects are related to its antioxidant effect.

Lycium barbarum extract has obvious antioxidant activity and has a protective effect on sperm. Lycium barbarum polysaccharide (LBP), the main effective component of Lycium barbarum, can promote the proliferation of spermatogonial stem cells in vitro, significantly increase the number of spermatogonial stem cells, and reduce the DNA damage of testicular cells induced by active oxygen free radicals through antioxidant effect.

Semen Allii Tuberosi extract can warm the kidney and help Yang, improve sexual function, and increase cold resistance, fatigue resistance and independent activity. When young male mice took Semen Allii Tuberosi extract, they could significantly increase their weight and the weight of testis, seminal vesicle gland and preputial gland.

Epimedii Folium has a long history of application as a Yang tonic. Modern pharmacological studies show that its main component, that is, icariin, is an inhibitor of PDE5, which can improve reproductive function. Icariin can increase the levels of dopamine and serotonin in hypothalamus, increase the concentration of testosterone and NO in blood, and promote the expression of eNOS, PI3K and phosphorylated Akt in penis. These results suggest that icariin can improve the sexual function of male mice through hypothalamic pituitary gonadal axis and PI3K/Akt/eNOS/NO signaling pathway. It is also reported that icariin can promote the proliferation of rat testicular cells through ERK1/2 signaling pathway. Chen et al. showed that icariin regulates peripheral benzodiazepine receptors and steroidogenic acute phase regulatory proteins to promote testosterone production. On the other hand, icariin also affects the expression of follicle stimulating hormone receptor in Sertoli cells and tight junction protein-11mRNA, reduces oxidative stress damage and improves male reproductive function.

Radix Morindae Officinalis is one of the important traditional Chinese medicines for tonifying the kidney and strengthening Yang. In recent years, researchers have done a lot of research on its role in improving reproductive function. It is found that Radix Morindae Officinalis oligosaccharides can significantly improve the sexual vitality and emergence rate of Drosophila melanogaster, and it has obvious kidney tonifying and Yang invigorating effects. Radix Morindae Officinalis aqueous extract, Radix Morindae Officinalis ethanol extract and Morinda officinalis total oligosaccharide crystallization interfered with the sperm reduction caused by cyclophosphamide in male mice. It was found that the three have obvious effects on promoting spermatogenesis. The water extract of Radix Morindae Officinalis can promote the repair of testis and spermatogenesis of mice irradiated by microwave. The alcohol extract of Radix Morindae Officinalis can improve the morphology of spermatogenic cells in damaged testes of mice. Wei Zhang and others found that Radix Morindae Officinalis can improve sperm activity, promote sperm production, play a positive role in sperm quality and quantity, and improve the reproductive ability of male animals

According to the efficacy analysis of the above-mentioned Chinese herbal medicines, in the combination of Semen Allii Tuberosi, Semen Cuscutae and Lycium barbarum: Semen Allii Tuberosi is the sovereign, which has the effect of warming the liver and kidney, invigorating Yang and consolidating essence, and is used for impotence and spermatorrhea caused by liver and kidney deficiency. Semen Cuscutae is the minister, which can help Semen Allii Tuberosi warming Yang, tonify liver and kidney, consolidate essence and shrink urine. Lycium barbarum is the assistant, which can balance Yin and Yang, nourish liver and kidney, benefit essence and replenish marrow.

In the combination of Semen Allii Tuberosi, Radix Morindae Officinalis and Epimedii Folium: Epimedii Folium is the sovereign, which can tonify kidney Yang, strengthen muscles and bones, treat kidney Yang deficiency, impotence, spermatorrhea and flaccid muscles and bones. Radix Morindae Officinalis is the minister, which can help the sovereign with tonifying the kidney Yang. It can also be used to warm liver and kidney, strengthen Yang and strengthen essence with Semen Allii Tuberosi as the assistant. The above three play a role in tonifying kidney and strengthening Yang.

In the combination of Semen Allii Tuberosi, Radix Morindae Officinalis and Cistanche deserticolaMa: Radix Morindae Officinalis, tonifying kidney Yang, strengthening muscles and bones, serving as the sovereign, is used for impotence, spermatorrhea, flaccid muscles and bones, etc. Cistanche deserticola Ma as the minister can not only help the sovereign to supplement the kidney Yang, but also benefit essence and blood, and cure deficiency of essence and blood. Semen Allii Tuberosi is the assistant, which can warm liver and kidney, strengthen Yang and strengthen essence. The above three are used together, which can tonify kidney and strengthen Yang, consolidate essence and lock Yang.

In addition, though the common dosage of each traditional Chinese medicine is known in the prior art, the prescription of the disclosure is made for the target disease by organically combining the above medicines, and its medicinal effect is not equivalent to the simple superposition of the effects of these medicines at a commonly-used amount. Actually, it cannot determine the amount of each medicine in the prescription according to their individual commonly-used amount, and the compounding ratio depends on many factors such as the characteristics of the medicinal materials and the compatibility of monarch drugs, ministerial drugs, adjuvant drugs and envoy drugs, and cannot be determined by experimental means such as comparison method and orthogonal test.

In an embodiment, the preparation method of the above traditional Chinese medicine compound composition with effect of improving male sexual function specifically includes the following steps.

(1) Semen Allii Tuberosi, Radix Morindae Officinalis or Lycium barbarum is subjected to extraction with water in a volume ratio of 1:8 under heating and refluxing for 3 h and filtration to obtain a residue. The residue is subjected to extraction twice, for 1.5 h and 1 h respectively. The filtrates are combined, concentrated, sprayed dry at 200° C. and sieved with a sieve of 80 mesh to obtain the corresponding extract.

(2) Epimedii Folium is extracted with 80% ethanol solution, concentrated and passed through the column to obtain an eluent. The eluent is concentrated, sprayed dry at 200° C. and sieved with a sieve of 80 mesh to obtain the corresponding extract.

(3) Cistanche deserticola Ma is extracted with 50% ethanol solution and filtered. The filtrates are combined, concentrated, sprayed dry at 200° C. and sieved with a sieve of 80 mesh to obtain the corresponding extract.

(4) Semen Cuscutae is extracted with 50% ethanol solution and filtered. The filtrates are combined, concentrated, sprayed dry at 200° C. and sieved with a sieve of 80 mesh to obtain the corresponding extract.

(5) The above extracts are mixed in a certain proportion to obtain compound A.

The compound A prepared by the above method can be made into capsules, tablets, granules and other dosage forms, and its application direction is to improve male sexual function.

Specifically, the disclosure also aims to provide an application of the traditional Chinese medicine compound composition with effect of improving male sexual function in the preparation of health food.

The compound composition disclosed by the disclosure can be used as a dietary supplement or health food raw material to improve male sexual dysfunction and easy fatigue.

It can be seen from the above technical scheme that, compared with the prior art, the disclosure provides a traditional Chinese medicine compound composition with effect of improving male sexual function and its application, which has the following excellent effects.

1) The disclosure can prepare the traditional Chinese medicine compound composition with effect of improving male sexual function by combining Semen Allii Tuberosi with Radix Morindae Officinalis, Epimedii Folium, Cistanche deserticola Ma, Lycium barbarum and Semen Cuscutae. The traditional Chinese medicine compound composition has a good effect on improving the sexual function of kidney Yang deficiency model mice. While improving their sexual function, it can improve the abundance of spermatogenic cells in testicular seminiferous tubules, ensure that spermatogenic cells can continuously produce new sperm, and improve the number and morphology of spermatozoa in the lumen.

2) The disclosure adopts the process of mixing the medicinal materials after separate extraction, which saves the extraction time and cost to the greatest extent, controls the main active substances of the medicinal materials, and ensures the stability and effectiveness of the products while following the economic principle.

3) In the prescription of the disclosure, Semen Allii Tuberosi, Radix Morindae Officinalis and Epimedii Folium are all raw materials that can be used in health food to improve male sexual dysfunction and easy fatigue. They are different from the hormone aphrodisiac products on the market, with no side effects, safety and easy absorption.

### BRIEF DESCRIPTION OF THE DRAWINGS

In order to explain the embodiments of the present disclosure or the technical solutions in the prior art more clearly, the following drawings that need to be used in the description of the embodiments or the prior art will be briefly introduced. Obviously, the drawings in the following description are only embodiments of the present disclosure. For those of ordinary skill in the art, other drawings can be obtained based on the drawings disclosed without creative work.

FIG. 1 shows the histomorphological observation of mouse testes (HE staining, ×100, ×400).

FIG. 2 is the histomorphological observation of mouse epididymides (HE staining, ×100, ×400).

FIG. 3 is the histomorphological observation of mouse kidneys (HE staining, ×100, ×400).

FIG. 4 is the histomorphological observation of mouse adrenal glands (HE staining, ×100, ×400).

### DETAILED DESCRIPTION OF THE EMBODIMENTS

Technical solutions of the present disclosure will be clearly and completely described below with reference to the embodiments. Obviously, described below are merely some embodiments of the disclosure, which are not intended to limit the disclosure. Other embodiments made by those skilled in the art without sparing any creative effort should fall within the scope of the disclosure.

The embodiments of disclosure provide a traditional Chinese medicine composition, which has the effect of improving male sexual function and can be used as a dietary supplement or health food raw material.

In order to better understand the present disclosure, the present disclosure will be further specifically described by the following embodiments, but it should not be construed as a limitation of the present disclosure. Some non-essential improvements and adjustments made by those skilled in the art according to the above-mentioned contents of the disclosure are also considered to fall within the protection scope of the present disclosure.

The disclosed technical solutions will be further described below with reference to specific embodiments.

### Embodiment 1

The raw materials of a traditional Chinese medicine compound composition for improving male sexual dysfunction and improving fatigue included 1 part of Semen Allii Tuberosi, 1 part of Lycium barbarum and 1 part of Semen Cuscutae.

The preparation method of the traditional Chinese medicine compound composition included the following steps.

(1) Semen Allii Tuberosi was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Semen Allii Tuberosi extract.

(2) Lycium barbarum was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Lycium barbarum extract.

(3) Semen Cuscutae was subjected to soak with 80% ethanol solution in a volume ratio of 1:8 for 0.5 h, extraction under heating and refluxing for 2 h and filtration to obtain a residue. The residue was subjected to extraction with 80% ethanol solution in a volume ratio of 1:6 under heating and refluxing twice, for 1 h each time. Then the extracting solutions were combined, purified, concentrated, sprayed dry at 200° C. and sieved with a sieve of 80 mesh to obtain the Semen Cuscutae extract.

The extracts obtained by the above preparation were uniformly mixed to obtain the traditional Chinese compound composition.

### Embodiment 2

The raw materials of a traditional Chinese medicine compound composition for improving male sexual dysfunction and improving fatigue included 2 parts of Semen Allii Tuberosi, 1 part of Epimedii Folium and 2 parts of Radix Morindae Officinalis.

The preparation method of the traditional Chinese medicine compound composition included the following steps.

(1) Semen Allii Tuberosi was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Semen Allii Tuberosi extract.

(2) Epimedii Folium was subjected to extraction with 80% ethanol solution in a volume ratio of 1:8 under heating and refluxing for 2 h and filtration to obtain a residue. The residue was subjected to extraction with 80% ethanol solution in a volume ratio of 1:6 under heating and refluxing for 2 times, 2 h each time. Then the extracting solutions were combined, passed through the column and concentrated to obtain eluents. The eluents were collected, concentrated, sprayed dry at 200° C. and sieved with a sieve of 80 mesh to obtain the Epimedii Folium extract.

(3) Radix Morindae Officinalis was subjected to extraction with 80% ethanol solution in a volume ratio of 1:8 under heating and refluxing for 2 h and filtration to obtain a residue. The residue was subjected to extraction with 80% ethanol solution in a volume ratio of 1:6 under heating and refluxing twice, for 2 h each time. Then the extracting solutions were combined, purified, concentrated, sprayed dry at 200° C. and sieved with a sieve of 80 mesh to obtain the Morindae Officinalis extract.

The extracts obtained by the above preparation were uniformly mixed to obtain the traditional Chinese compound composition.

### Embodiment 3

The raw materials of a traditional Chinese medicine compound composition for improving male sexual dysfunction and improving fatigue included 2 parts of Semen Allii Tuberosi, 1 part of Epimedii Folium and 1 part of Radix Morindae Officinalis.

The preparation method of the traditional Chinese medicine compound composition included the following steps.

(1) Semen Allii Tuberosi was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Semen Allii Tuberosi extract.

(2) Cistanche deserticola Ma was subjected to extraction with 50% ethanol solution in a volume ratio of 1:8 under heating and refluxing for 2 h and filtration to obtain a residue. The residue was subjected to extraction with 50% ethanol solution in a volume ratio of 1:6 under heating and refluxing twice, for 2 h each time. Then the extracting solutions were combined, concentrated, sprayed dry at 200° C. and sieved with a sieve of 80 mesh to obtain the Cistanche deserticola Ma extract.

(3) Radix Morindae Officinalis was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 2 h each time. Then the extracting solutions were combined, purified, concentrated, sprayed dry at 200° C. and sieved with a sieve of 80 mesh to obtain the Morindae Officinalis extract.

The extracts obtained by the above preparation were uniformly mixed to obtain the traditional Chinese compound composition.

### Embodiment 4

Preparation of Capsules:

The raw materials of a traditional Chinese medicine compound composition for improving male sexual dysfunction and improving fatigue included 2 parts of Semen Allii Tuberosi, 1 part of Epimedii Folium, 1 part of Radix Morindae Officinalis, 2 parts of Lycium barbarum, 2 parts of Semen Cuscutae, and 1 part of Cistanche deserticola Ma.

The preparation method of the traditional Chinese medicine compound composition included the following steps.

(1) Semen Allii Tuberosi was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Semen Allii Tuberosi extract.

(2) Epimedii Folium was subjected to extraction with 80% ethanol solution in a volume ratio of 1:8 under heating and refluxing for 2 h and filtration to obtain a residue. The residue was subjected to extraction with 80% ethanol solution in a volume ratio of 1:6 under heating and refluxing twice, for 2 h each time. Then the extracting solutions were combined, passed through the column and concentrated to obtain eluents. The eluents were collected, concentrated, sprayed dry at 200° C. and sieved with a sieve of 80 mesh to obtain the Epimedii Folium extract.

(3) Cistanche deserticola Ma was subjected to extraction with 50% ethanol solution in a volume ratio of 1:8 under heating and refluxing for 2 h and filtration to obtain a residue. The residue was subjected to extraction with 50% ethanol solution in a volume ratio of 1:6 under heating and refluxing twice, for 2 h each time. Then the extracting solutions were combined, concentrated, sprayed dry at 200° C. and sieved with a sieve of 80 mesh to obtain the Cistanche deserticola Ma extract.

(4) Lycium barbarum was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the filtrates were combined, purified, concentrated, sprayed dry at 200° C. and sieved with a sieve of 80 mesh to obtain the Lycium barbarum extract.

(5) Semen Cuscutae was subjected to soak with 80% ethanol solution in a volume ratio of 1:8 for 0.5 h, extraction under heating and refluxing for 2 h and filtration to obtain a residue. The residue was subjected to extraction with 80% ethanol solution in a volume ratio of 1:6 under heating and refluxing twice, for 1 h each time. Then the filtrates were combined, purified, concentrated, sprayed dry at 200° C. and sieved with a sieve of 80 mesh to obtain the Semen Cuscutae extract.

(6) Radix Morindae Officinalis was subjected to extraction with 80% ethanol solution in a volume ratio of 1:8 under heating and refluxing for 2 h and filtration to obtain a residue. The residue was subjected to extraction with 80% ethanol solution in a volume ratio of 1:6 under heating and refluxing twice, for 2 h each time. Then the extracting solutions were combined, passed through the column and concentrated to obtain eluents. The eluents were collected, concentrated, sprayed dry at 200° C. and sieved with a sieve of 80 mesh to obtain the Radix Morindae Officinalis extract.

The extracts prepared above were mixed according to the ratio of Semen Allii Tuberosi: Lycium barbarum: Semen Cuscutae (18:10:72) to obtain mixture A, Semen Allii Tuberosi: Epimedii Folium: Radix Morindae Officinalis (18:10:72) to obtain mixture B, and Semen Allii Tuberosi: Cistanche deserticola Ma: Radix Morindae Officinalis (20:45:35) to obtain mixture C.

Pregelatinized starch, talc powder and magnesium stearate were added into the prepared traditional Chinese medicine compounds, then mixed evenly, and put into capsules to prepare capsules.

### Embodiment 5

Preparation of Tablets:

The raw materials of a traditional Chinese medicine compound composition for improving male sexual dysfunction and improving fatigue included 2 parts of Semen Allii Tuberosi, 1 part of Epimedii Folium, 1 part of Radix Morindae Officinalis, 2 parts of Lycium barbarum, 2 parts of Semen Cuscutae, and 1 part of Cistanche deserticola Ma.

The preparation method of the traditional Chinese medicine compound composition included the following steps.

(1) Semen Allii Tuberosi was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Semen Allii Tuberosi extract.

(2) Epimedii Folium was subjected to extraction with 80% ethanol solution in a volume ratio of 1:8 under heating and refluxing for 2 h and filtration to obtain a residue. The residue was subjected to extraction with 80% ethanol solution in a volume ratio of 1:6 under heating and refluxing twice, for 2 h each time. Then the extracting solutions were combined, passed through the column and concentrated to obtain eluents. The eluents were collected, concentrated, sprayed dry at 200° C. and sieved with a sieve of 80 mesh to obtain the Epimedii Folium extract.

(3) Cistanche deserticola Ma was subjected to extraction with 50% ethanol solution in a volume ratio of 1:8 under heating and refluxing for 2 h and filtration to obtain a residue. The residue was subjected to extraction with 50% ethanol solution in a volume ratio of 1:6 under heating and refluxing twice, for 2 h each time. Then the extracting solutions were combined, concentrated, sprayed dry at 200° C. and sieved with a sieve of 80 mesh to obtain the Cistanche deserticola Ma extract.

(4) Lycium barbarum was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Lycium barbarum extract.

(5) Semen Cuscutae was subjected to soak with 80% ethanol solution in a volume ratio of 1:8 for 0.5 h, extraction under heating and refluxing for 2 h and filtration to obtain a residue. The residue was subjected to extraction with 80% ethanol solution in a volume ratio of 1:6 under heating and refluxing twice, for 1 h each time. Then the extracting solutions were combined, purified, concentrated, sprayed dry at 200° C. and sieved with a sieve of 80 mesh to obtain the Semen Cuscutae extract.

(6) Radix Morindae Officinalis was subjected to extraction with 80% ethanol solution in a volume ratio of 1:8 under heating and refluxing for 2 h and filtration to obtain a residue. The residue was subjected to extraction with 80% ethanol solution in a volume ratio of 1:6 under heating and refluxing twice, for 2 h each time. Then the extracting solutions were combined, passed through the column and concentrated to obtain eluents. The eluents were collected, concentrated, sprayed dry at 200° C. and sieved with a sieve of 80 mesh to obtain the Radix Morindae Officinalis extract.

The extracts prepared above were mixed according to the ratio of Semen Allii Tuberosi: Lycium barbarum: Semen Cuscutae (18:10:72) to obtain mixture A, Semen Allii Tuberosi: Epimedii Folium: Radix Morindae Officinalis (18:10:72) to obtain mixture B, and Semen Allii Tuberosi: Cistanche deserticola Ma: Radix Morindae Officinalis (20:45:35) to obtain mixture C.

Pregelatinized starch, talc powder and magnesium stearate were added to the prepared traditional Chinese medicine compounds, then mixed evenly, pressed into tablets, coated with film, and made into tablets.

### Embodiment 6

Preparation of Granules:

The raw materials of a traditional Chinese medicine compound composition for improving male sexual dysfunction and improving fatigue included 2 parts of Semen Allii Tuberosi, 1 part of Epimedii Folium, 1 part of Radix Morindae Officinalis, 2 parts of Lycium barbarum, 2 parts of Semen Cuscutae, and 1 part of Cistanche deserticola Ma.

The preparation method of the traditional Chinese medicine compound composition included the following steps.

(1) Semen Allii Tuberosi was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Semen Allii Tuberosi extract.

(2) Epimedii Folium was subjected to extraction with 80% ethanol solution in a volume ratio of 1:8 under heating and refluxing for 2 h and filtration to obtain a residue. The residue was subjected to extraction with 80% ethanol solution in a volume ratio of 1:6 under heating and refluxing twice, for 2 h each time. Then the extracting solutions were combined, passed through the column and concentrated to obtain eluents. The eluents were collected, concentrated, sprayed dry at 200° C. and sieved with a sieve of 80 mesh to obtain the Epimedii Folium extract.

(3) Cistanche deserticola Ma was subjected to extraction with 50% ethanol solution in a volume ratio of 1:8 under heating and refluxing for 2 h and filtration to obtain a residue. The residue was subjected to extraction with 50% ethanol solution in a volume ratio of 1:6 under heating and refluxing twice, for 2 h each time. Then the extracting solutions were combined, concentrated, sprayed dry at 200° C. and sieved with a sieve of 80 mesh to obtain the Cistanche deserticola Ma extract.

(4) Lycium barbarum was subjected to extraction with water in a volume ratio of 1:8 under heating and refluxing for 2 h and filtration to obtain a residue. The residue was subjected to extraction with deionized water in a volume ratio of 1:6 under heating and refluxing twice, for 1 h each time. Then the extracting solutions were combined, purified, concentrated, sprayed dry at 200° C. and sieved with a sieve of 80 mesh to obtain the Lycium barbarum extract.

(5) Semen Cuscutae was subjected to soak with 80% ethanol solution in a volume ratio of 1:8 for 0.5 h, extraction under heating and refluxing for 2 h and filtration to obtain a residue. The residue was subjected to extraction with 80% ethanol solution in a volume ratio of 1:6 under heating and refluxing twice, for 1 h each time. Then the filtrates were combined, purified, concentrated, sprayed dry at 200° C. and sieved with a sieve of 80 mesh to obtain the Semen Cuscutae extract.

(6) Radix Morindae Officinalis was subjected to extraction with 80% ethanol solution in a volume ratio of 1:8 under heating and refluxing for 2 h and filtration to obtain a residue. The residue was subjected to extraction with 80% ethanol solution in a volume ratio of 1:6 under heating and refluxing twice, for 2 h each time. Then the extracting solutions were combined, passed through the column and concentrated to obtain eluents. The eluents were collected, concentrated, sprayed dry at 200° C. and sieved with a sieve of 80 mesh to obtain the Radix Morindae Officinalis extract.

The extracts prepared above were mixed according to the ratio of Semen Allii Tuberosi: Lycium barbarum: Semen Cuscutae (18:10:72) to obtain mixture A, Semen Allii Tuberosi: Epimedii Folium: Radix Morindae Officinalis (18:10:72) to obtain mixture B, and Semen Allii Tuberosi: Cistanche deserticola Ma: Radix Morindae Officinalis (20:45:35) to obtain mixture C.

(7) The above mixture A, B or C was mixed with a certain proportion of lactose monohydrate evenly, and 95% ethanol was added to produce a mixture. The mixture was stirred until it was kneaded into a dough and dispersed when touched, and then granulation was started. Granules that can not pass through the 20-mesh sieve and 80-mesh sieve but can pass the 10-mesh sieve were collected and dried in an oven in time for use.

(8) The prepared granules were put into a vacuum dryer for drying so that the moisture content of the granules was controlled at 4%. The compound granules with the effect of improving male sexual dysfunction and fatigue were prepared.

The content of the disclosure is not limited to the above embodiments, and one or the combination of several embodiments can also achieve the purpose of the disclosure.

Further, the following test was performed to evaluate the efficacy of the traditional Chinese medicine compound composition prepared herein.

### Efficacy Test

### I. Test Purpose

The Efficacy test was carried out to explore the effects of several traditional Chinese medicines to be studied on the sexual function of kidney Yang deficiency model mice induced by hydrocortisone, to observe the effects of the above traditional Chinese medicines on the general external performance, sexual behavior (capture, mount, ejaculation latency and times), kidney, adrenal gland, testis, epididymis and seminal vesicle organ index, tissue morphology, and various sex hormone levels in serum of the kidney Yang deficiency mice, to explore the mechanism of the above-mentioned traditional Chinese medicines to improve the sexual function of kidney Yang deficiency model mice, so as to provide data support for the prevention and treatment of sexual dysfunction of kidney Yang deficiency mice by relevant traditional Chinese medicines.

### II. Test materials and methods

### 1. Animals, Reagents and Instruments

### 1.1 Animals

100 male and 100 female clean ICR mice, 4-6 weeks old, 18-22 g, were purchased from SiPeiFu (Beijing) Biotechnology Co., Ltd. with certificate No. SCXK (Beijing) 2019-0010. All tests of the disclosure were carried out in accordance with the ethical code of experimental animals.

### 1.2 Drugs and Reagents

TABLE 1
Test drugs and reagents

| Reagent name | Manufactor | Article Number |
|---|---|---|
| Hydrocortisone injection | Changzhou SiYao | License No.: |
| | Pharmaceutical | GUOYAOZHUNZI |
| | Co., Ltd | H20093293 |
| Estradiol valerate tablets | Bayer Healthcare | License No.: |
| | Company Limited | GUOYAOZHUNZI |
| | Guangzhou Branch | [symbol] |
| Testosterone (T) ELISA Kit | Beijing GongGeBoYe | DG30457M-96T, |
| | Biotechnology | [symbol] |
| | Co., Ltd | |
| Folliclestimulating | Beijing GongGeBoYe | DG30497M-96T, |
| Hormone | Biotechnology | DG30497M-48T |
| (FSH) ELISA Kit | Co., Ltd | |
| Luteinizing Hormone (LH) | Beijing GongGeBoYe | DG30426M-96T, |
| ELISA Kit | Biotechnology | DG30426M-48T |
| | Co., Ltd | |
| Superoxidedismutase | Nanjing Jiancheng | A001-3-1, |
| (SOD) | Bioengineering | A001-3-2 |
| Biochemical Kit | Institute | |
| Mmalondialdehyde (MDA) | Nanjing Jiancheng | A003-1-1, |
| Biochemical Kit | Bioengineering | A003-1-2 |
| | Institute | |
| Glutathion peroxidase | Nanjing Jiancheng | A005-1-2 |
| (GSH-PX) | Bioengineering | |
| Assay Kit | Institute | |
| Nitricoxidesynthase | Nanjing Jiancheng | A014-2-1, |
| (NOS) | Bioengineering | A014-2-2 |
| Biochemical Kit | Institute | |

[symbol]

indicates data missing or illegible when filed

### 1.3 Test Instrument

TABLE 2
Test instrument

| Name | Manufactor | Model |
|---|---|---|
| Centrifuge | Beckman Coulter | AllgraX-15R |
| Microplate Reader | BioTek | Biotek Epoch2 |
| Sartorius Electronic | Sartorius | BP211D |
| Analytical Balance | | |
| Dehydrator | Wuhan Junjie Electronics Co., Ltd | JJ-12J |
| Embedding machine | Wuhan Junjie Electronics Co., Ltd | JB-P5 |
| Pathological microtome | Shanghai Leica Instrument Co., Ltd | RM2016 |
| Freezing table | Wuhan Junjie Electronics Co., Ltd | JB-L5 |
| Tissue spreader | Zhejiang Jinhua Kedi Instrument | KD-P |
| | Equipment Co., Ltd | |
| Oven | Tianjin Labotery Instrument | GFL-230 |
| | Equipment Co., Ltd | |
| Glass slide | Servicebio | — |
| Orthostatic optical | Nikon | Nikon Eclipse E100 |
| microscope | | |
| Imaging system | Nikon | NIKON DS-U3 |

### 2. Model Establishment and Group Administration

Male mice were randomly divided into normal group, model group, sample 1 group, sample 2 group, sample 3 group and sample 4 group. Each administration group was given the corresponding dose by gavage. The normal group and the model group were given the same volume of normal saline (1 mL/10 g) by gavage once a day for 4 weeks. The model was established on the 7th day of the second week after gavage. Except the normal group, the kidney Yang deficiency model was established by injecting hydrocortisone (37.5 mg/kg/d) intramuscularly for 14 days. At the same time, the model was continuously administered. On the 7th day of the 4th week, the general external performance and behavioral performance of the animals were observed and recorded one hour after administration, and then the mice were anesthetized and the test materials were taken.

TABLE 3
Test grouping and dosage

| Group | Tab | Dosage |
|---|---|---|
| Normal group | Normal | / |
| Model group | Model | / |
| Compound of Semen Allii Tuberosi, | Sample 1 | 200 mg/kg |
| Lycium barbarum and Semen Cuscutae | | |
| Compound of Semen Allii Tuberosi, | Sample 2 | 200 mg/kg |
| Radix Morindae Officinalis and Epimedii Folium | | |
| Compound of Semen Allii Tuberosi, | Sample 3 | 200 mg/kg |
| Radix Morindae Officinalis and | | |
| Cistanche deserticola Ma | | |
| Positive control - Aphrodyne Tablets | Sample 4 | 83 mg/kg |

### 3. Index Detection

### (1) General External Performance

The weight, body temperature, hair color and shape, eating state and other main performance of male mice before and after treatment were observed and recorded.

### (2) Sexual Behavior of Male Mice Observed by Mating Test

On the 7th day of the 4th week of administration of male mice, all animals were subjected to mating experiments according to groups. Female mice were given estradiol valerate (1 mg/kg) by gavage once a day on the 24th to 27th days to synchronize the female mice with estrus. The male mice caged with their corresponding female mice 1 h after the last administration. The specific operations were as follows. The male mice were put in each group separately into the cage. The surrounding environment was kept dark and quiet. The red dim light was used to make them adapt to the environment for 5mins, and then the corresponding female mice were put into the cage for cage combining test. The following indexed were observed within 30 mins of cage combining.

a. Capture latency (CL): the time from the female mouse being put into the observation box to the male mouse capturing the female mouse for the first time (unit: s). Capture behavior: the behavior of catching and chasing the female mouse after the male mouse sniffed the base of the female mouse's tail.

b. Capture times (CT): the number of times that male mouse captured female mouse within 30 min.

c. Mount latency (ML): the time interval from when the female mouse was put into the observation box to the first mount behavior (unit: s). Mount behavior: the sexual behavior of the male mouse's waist doing back and forth motions, while the hind limbs repeatedly do flexion and extension movements, with or without insertion counted.

d. Mount times: the number of mount times before male mouse ejaculated.

e. Ejaculation latency: time interval from the time the female mouse was put into the observation box to the first ejaculation of the male mouse (unit: s).

f. Ejaculation times (ET).

### (3) Organ Index Determination

24 h after the last administration, the male mice were weighed and killed. The kidneys, adrenal glands, testes, epididymides and seminal vesicles of the male mice were quickly taken out, weighed accurately, and the organ index was calculated, where organ index=organ weight (mg)/body mass of the mouse (g).

### (4) HE Staining for Observing Histomorphology of Kidneys, Adrenal Glands, Testes and Epididymides

The kidneys, adrenal glands, testes and epididymides of male mice were fixed with 10% formalin. The tissues were dehydrated, embedded in wax and sectioned. Five of them were taken from each group for HE staining.

1) Dewaxing paraffin sections to water: the sections were successively placed in xylene I for 20 min—xylene II for 20 min—anhydrous ethanol I for 5 min—anhydrous ethanol II for 5 min—75% ethanol solution for 5 min, and washed with tap water.

2) Hematoxylin staining: the sections were stained with hematoxylin staining solution for 3-5 min, washed with tap water, differentiated with differentiation solution, washed with tap water, promoted to blue with blue promoting solution, and rinsed with running water.

3) Eosin staining: the sections were dehydrated with 85% and 95% gradient ethanol solution for 5 min respectively, and stained with eosin staining solution for 5 min.

4) Dehydration sealing: the sections were successively placed in anhydrous ethanol I for 5 min—anhydrous ethanol II for 5 min—anhydrous ethanol III for 5 min—xylene I for 5 min—xylene II for 5 min in order to be transparent, and sealed by neutral gum.

5) Microscopic examination, image acquisition and analysis, pathological observation of tissue morphology and magnification of corresponding times (100×˜400×) for taking photos were performed successively.

### (5) ELISA to Detect the Levels of Various Sex Hormones in Serum

Orbital blood was collected from male mice, left standing for 1 h, centrifuged at 3000 r/min for 10 min to separate serum, and the levels of serum T, E2, FSH, LH and GnRH were determined by Elisa method according to the kit instructions.

### (6) Detection of Peroxide Level in Testes by Biochemical Method

The testis tissues of male mice were frozen and stored in −80° C. refrigerator. The tissues were ground when used, and the expressions of testis SOD, MDA, GSH-PX and penile NOS were detected according to the instructions of the biochemical kit.

### 4. Statistical Analysis

SPSS 19.0 software was used for statistics, measurement data were expressed as mean±standard deviation (mean±SD), and one-way-ANOVA was used to compare more than two groups. P<0.05 means the difference was statistically significant.

### III. Test Results

### 1. General State

Compared with the normal mice, the mice in the model group had kidney Yang deficiency symptoms, such as aversion to cold, decreased autonomous activity, dull fur, and some of the mice were depressed in mental state and slow in activity. The general state of mice in each sample group was improved to some extent.

### 2. Sexual Behavior

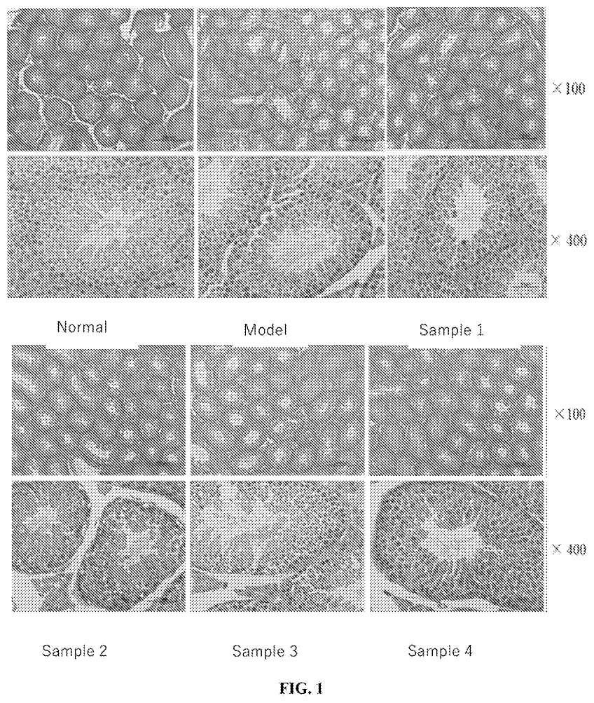

Compared with the normal group, the capture times, mount times and ejaculation times of the model group were significantly reduced (P<0.05). Compared with the model group, the capture latency of sample 1 group was shorter, and the capture times, mount times and ejaculation times were significantly increased (P<0.05), the capture times of sample 2 group and sample 4 group were significantly increased (P<0.05), the capture times, mount times and ejaculation times of sample 4 group were significantly increased (P<0.05), and the capture latency of sample 4 group was shorter, and the capture times of sample 7 group were significantly increased (P<0.05), which were shown in Table 4 and FIG. 1. The results suggest that sample 2 and sample 4 can improve the sexual behavior of kidney Yang deficiency model mice.

TABLE 4
Comparison of sexual behavior of mice in each group after administration (\grave{ }x ± s)

| Group | Normal | Model | Sample 1 | Sample 2 | Sample 3 | Sample 4 |
|---|---|---|---|---|---|---|
| CL(s) | 7.00 ± 4.69 | 8.42 ± 7.81 | 6.70 ± 2.45 | 8.00 ± 2.00 | 6.50 ± 3.21 | 5.40 ± 2.22 |
| CT (times) | 39.60 ± 21.46 | 16.58 ± 6.52* | 24.70 ± 10.06 | 27.60 ± 9.67# | 24.30 ± 9.23 | 36.80 ± 14.99# |
| ML(s) | 205.00 ± 124.72 | 254.73 ± 321.17 | 158.70 ± 148.63 | 171.70 ± 106.92 | 185.22 ± 100.39 | 145.70 ± 131.50 |
| MT (times) | 18.60 ± 12.30 | 8.36 ± 6.79* | 13.00 ± 5.87 | 13.80 ± 4.69 | 14.44 ± 5.48 | 18.40 ± 9.03# |
| EL(s) | 328.43 ± 125.57 | 364.11 ± 101.73 | 268.50 ± 90.41 | 278.33 ± 82.99 | 250.00 ± 101.09 | 256.25 ± 124.14 |
| ET (times) | 8.57 ± 5.91 | 3.67 ± 2.83* | 4.25 ± 2.92 | 5.00 ± 2.87 | 5.33 ± 3.44 | 8.63 ± 3.54# |

Note:
*compared with the normal group, P < 0.05. #Compared with the model group, P < 0.05.

### 3. Tissue Morphology

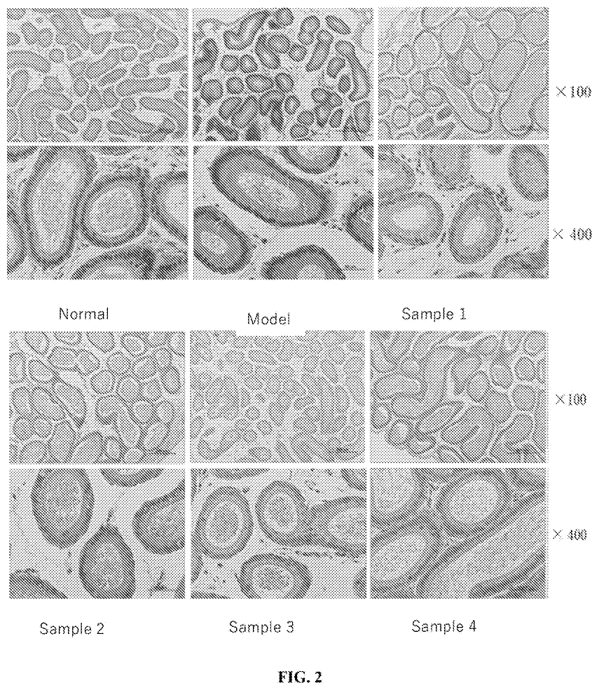

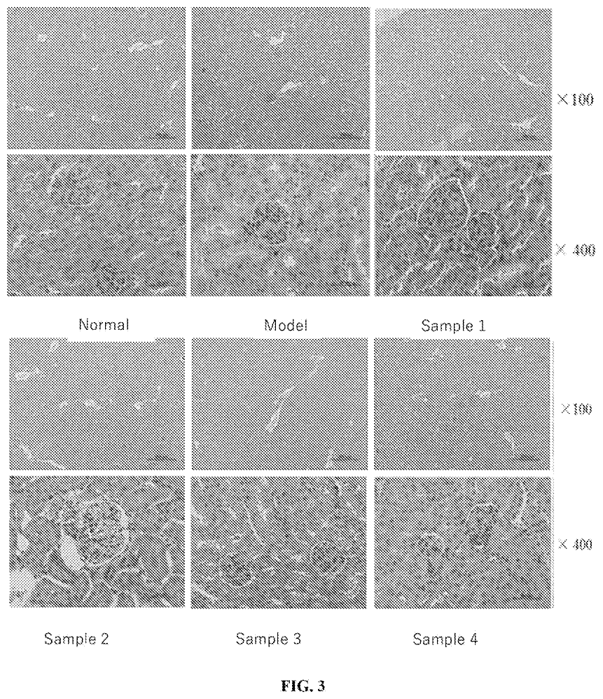

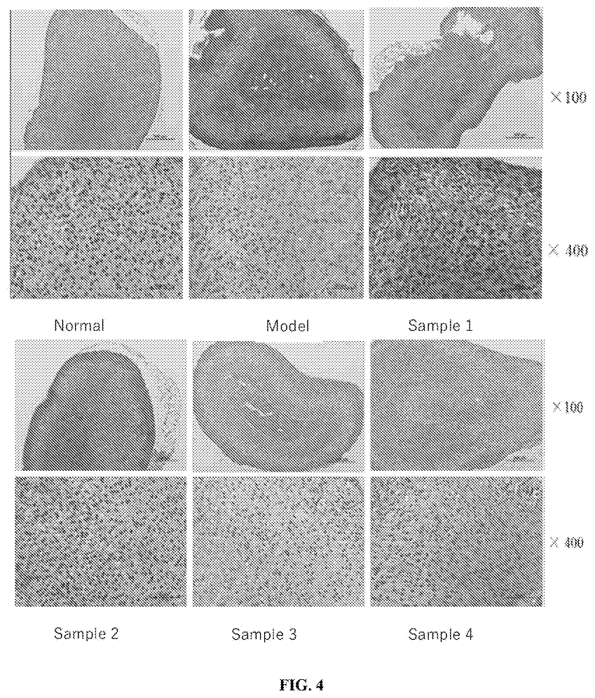

The tissue structure of each specimen of normal mice was normal. There were abundant layers of spermatogenic cells in the seminiferous tubule of testes, sperm were found in the lumen, and the epididymal tubes were closely arranged with abundant sperm in the tubule. Although all layers of spermatogenic cells in the seminiferous tubules of the testes of the model group were visible, the local layers were reduced, no intact sperm was found in the lumen, denatured sperm were visible, the arrangement of epididymal tubules was loose, and the number of sperms in the tubules was reduced. After administration, the layers of testicular seminiferous epithelium of mice in each group increased in varying degrees, the number of sperms cells and sperm increased, the arrangement of epididymal tubes improved compared with the model group, and the number of sperms in the lumen increased. In the sample 2 group and sample 4 group, the epithelium of testicular seminiferous tubules was significantly improved, spermatogenic cells in all layers were visible, the epididymides were closely arranged, and the number of sperms in the lumen was increased. No obvious pathological changes were found in adrenal histomorphology of mice in each group, as shown in FIG. 2 to FIG. 4.

### 4. Sex Hormones

Compared with the normal group, the levels of T, E2, FSH, LH and GnRH in the model group decreased (P<0.05). Compared with the model group, the levels of serum T, E2, FSH, LH and GnRH of mice in sample 4 group increased significantly (P<0.05), the levels of T, E2, FSH and GnRH in sample 2 group increased significantly (P<0.05), the levels of LH, T and E2 in sample 1 group increased (P<0.05), and the levels of FSH, T and E2 in sample 3 group increased (P<0.05), as shown in Table 5. The results showed that each sample had different regulating effects on the level of sex hormones, and sample 2 and sample 4 had the most obvious effect on the improvement of sex hormones in kidney Yang deficiency model mice.

TABLE 5
Levels of sex hormones in serum of mice in each group after
administration (\grave{ } x ± s)

| | T | E2 | FSH | LH | GnRH |
|---|---|---|---|---|---|
| Group | (ng/mL) | ( [symbol] | (mIU/mL) | (mU/mL) | (mIU/mL) |
| Normal | 3.42 ± 0.42 | 6.97 ± 1.49 | 9.81 ± 1.84 | 4.65 ± 0.79 | 13.46 ± 2.38 |
| Model | 2.81 ± 0.61* | 5.26 ± 0.96* | 7.93 ± 1.95* | 3.69 ± 0.66* | 11.32 ± 1.95* |
| Sample 1 | 3.28 ± 0.46# | 6.66 ± 0.62# | 9.35 ± 1.69 | 4.60 ± 0.52# | 13.04 ± 1.80 |
| Sample 2 | 3.45 ± 0.59# | 7.18 ± 0.86# | 9.71 ± 1.50# | 4.32 ± 0.71 | 14.10 ± 0.37# |
| Sample 3 | 3.39 ± 0.49# | 7.06 ± 1.20# | 9.81 ± 1.49# | 4.25 ± 1.03 | 13.05 ± 1.77 |
| Sample 4 | 3.46 ± 0.59# | 7.25 ± 0.86# | 10.33 ± 1.18# | 4.60 ± 0.52# | 14.28 ± 0.43# |

Note:
*Compared with the normal group, P<0.05.
#Compared with the model group, P < 0.05. [symbol]
indicates data missing or illegible when filed

### 5. Levels of Oxidative Stress Related Factors

Compared with the normal group, the testicular SOD decreased, MDA increased, and the penile NOS activity decreased in the model group (P<0.05). Compared with the model group, the testicular SOD and GSH-Px activities in sample 2 group and sample 4 group increased significantly (P<0.05), and penile NOS activities in sample 1 group and sample 4 group increased significantly (P<0.05), as shown in Table 6. The results showed that sample 2 and 4 could improve the oxidative damage of testes, and sample 1 and 4 could also improve the erectile function of penises.

TABLE 6
Levels of oxidative stress related factors in testes and penises of mice
in each group after administration (\grave{ } x±s)

| | Testicular SOD | Testicular MDA | Testicular | |
|---|---|---|---|---|
| | activity | content | GSH-Px | Penile NOS |
| Group | (U/mgprot) | (nmol/mgprot) | activity | (U/mgprot) |
| Normal | 131.78 ± 35.76 | 5.57 ± 1.52 | 136.87 ± 38.75 | 2.20 ± 0.88 |
| Model | 82.47 ± 7.23* | 7.03 ± 1.23* | 94.97 ± 23.64* | 0.80 ± 0.40* |
| Sample 1 | 81.83 ± 9.09 | 6.13 ± 0.96 | 128.11 ± 51.79 | 2.1 ± 1.27# |
| Sample 2 | 104.98 ± 17.47# | 6.08 ± 1.05 | 137.47 ± 59.37# | 1.48 ± 0.50 |
| Sample 3 | 85.85 ± 4.67 | 6.72 ± 1.17 | 130.96 ± 72.67 | 1.00 ± 0.77 |
| Sample 4 | 102.27 ± 11.04# | 6.17 ± 1.41 | 146.40 ± 42.26# | 2.47 ± 1.57# |

Note:
*compared with the normal group, P < 0.05.
#compared with the model group, P < 0.05.

### IV. Conclusion

The test results of the disclosure show that, compared with the normal group, the capture times, mount times and ejaculation times of the model group mice were significantly reduced, the testicular index was significantly reduced, the levels of serum GnRH, T, E2, FSH and LH were reduced, the testicular SOD and GSH-Px were reduced, MDA was increased, and the penis NOS activity was reduced. Compared with the model group, the levels of LH, T and E2 in sample 1 group were increased, and the penile NOS activity was increased. The capture times of sample 2 group were significantly increased, the levels of serum GnRH, FSH, T and E2 were significantly increased, and the testicular SOD activity and GSH-Px activity were significantly increased. The levels of serum FSH, T and E2 in sample 3 group were increased. The capture times, mount times and ejaculation times, the testicular index, the levels of serum GnRH, T, E2, FSH and LH, the testicular SOD activity, GSH-Px activity and penile NOS activity were significantly increased in the sample 4 group.

Mouse mating test is a common method to evaluate sexual function. The latency and frequency of sexual behaviors such as capture, mount, and ejaculation can reflect the strength of sexual function. When one of the results is positive, it indicates that the drug has a certain promoting effect on sexual function. The test of the disclosure finds that sample 2 and sample 4 can increase the capture times of kidney Yang deficiency model mice. It can be seen that sample 2 and sample 4 can improve the mating ability and sexual function of mice. They are good sexual function enhancers and can improve the sexual desire and sexual activity ability of mice to a certain extent.

Atrophy and pathological changes of reproductive organs can lead to sexual dysfunction to some extent. The results of HE staining showed that the epithelium of seminiferous tubules in the testes of the sample 2 group was significantly improved, spermatogenic cells in all layers were visible, the epididymides were closely arranged, and the number of sperms in the lumen was increased. Therefore, sample 2 can improve the pathological changes of testes.

Sexual behavior is regulated by sex hormones, and the hypothalamus-pituitary-gonad axis (HPG axis) is the main regulatory pathway in the body. GnRH is synthesized in hypothalamus, which promotes pituitary gonadotropin cells to secrete LH and FSH, and then regulates the synthesis and secretion of sex hormones T and E2, while sex hormones reversely regulate pituitary and hypothalamus to form a feedback pathway to maintain endocrine homeostasis. The test of the disclosure shows that sample 2 can increase the levels of GnRH, FSH, T and E2, sample 1 can increase the levels of LH, T and E2, and sample 3 can increase the levels of FSH, T and E2, suggesting that each sample has the effect of regulating the serum sex hormones of kidney Yang deficiency model mice to varying degrees. Sample 2 may improve sexual function by regulating hypothalamic-pituitary-gonadal axis function, mainly acting on FSH. Sample 1 mainly acts on LH. Sample 3 mainly acts on FSH. LH and FSH mainly coregulate a series of processes related to male growth, development, puberty sexual maturity and reproduction. Among them, LH mainly stimulates interstitial cells to synthesize and secrete T, providing a hormone environment for spermatogenesis. FSH mainly promotes spermatogenesis and germ cell differentiation. Therefore, sample 1 focuses on stimulating the synthesis and secretion of T by interstitial cells, and samples 2 and 3 focus on promoting spermatogenesis and germ cell differentiation.

NOS is the rate limiting enzyme for the synthesis of nitric oxide (NO), and its activity changes have a great impact on the functional state of the neuroendothelial dependent NO erectile system. If the activity of NOS in penile cavernosum tissue decreases, it will inevitably affect the normal play of erectile function. It is found in the test of the disclosure that sample 1 can increase the penile NOS activity, suggesting that these samples can improve the erectile function of the penis.

In the current research on male infertility, the view that oxidative stress is the cause of infertility is recognized by most scholars. When the strong oxidants produced in the body are too much and exceed the normal scavenging capacity of the antioxidant enzymes in the body under the original physiological state, the physiological function of the body's normal antioxidation will be abnormal, resulting in tissue damage. SOD and GSH-Px are important antioxidant enzymes to eliminate oxidants in the body. They are also important substances to enhance immunity and prevent diseases in the human body. They can remove free radicals and protect cells from oxidative damage. MDA is the oxidation end product of the oxidation reaction of lipids by free radicals in living organisms, and its content can reflect the degree of oxidative stress damage. The test of the disclosure finds that sample 2 can increase the testicular SOD activity and GSH-Px activity, reduce oxidative stress damage by enhancing the ability to scavenge oxidative free radicals in mice, reduce MDA level, and has better effect in improving oxidative stress damage.

In conclusion, different samples can improve male sexual function to varying degrees. Sample 1, Sample 2 and Sample 3 can improve the sexual behavior, serum sex hormones and testicular antioxidant capacity of kidney Yang deficiency model mice in varying degrees, and can improve the level of sex hormones by inhibiting oxidative stress, so as to improve male sexual function.

The above description of the disclosed embodiments enables the skilled in the art to achieve or use the disclosure. Multiple modifications to these embodiments will be apparent to those skilled in the art, and the general principles defined herein may be achieved in other embodiments without departing from the spirit or scope of the disclosure. The present disclosure will therefore not be restricted to these embodiments shown herein, but rather to comply with the broadest scope consistent with the principles and novel features disclosed herein.

What is claimed is:

1. A traditional Chinese medicine compound composition with effect of improving male sexual function, wherein the traditional Chinese medicine compound composition is prepared by mixing following raw materials after separate extraction, and the raw materials comprises:
1˜10 parts by weight of Semen Allii Tuberosi;
1˜10 parts by weight of Radix Morindae Officinalis;
1˜10 parts by weight of Epimedii Folium;
or 1˜10 parts by weight of Semen Allii Tube rosi;
1˜10 parts by weight of Radix Morindae Officinalis;
1˜10 parts by weight of Cistanche deserticola Ma;
or 1˜10 parts by weight of Semen Allii Tuberosi;
1˜10 parts by weight of Lycium barbarum;
1˜10 parts by weight of Semen Cuscutae;
or 1˜10 parts by weight of Semen Allii Tuberosi;
1˜10 parts by weight of Lycium barbarum;
1˜10 parts by weight of Semen Cuscutae;
1˜10 parts by weight of Radix Morindae Officinalis;
1˜10 parts by weight of Cistanche deserticola Ma;
1˜10 parts by weight of Epimedii Folium.

2. An application of the traditional Chinese medicine compound composition with effect of improving male sexual function of claim 1 in a preparation of health food.

3. The application of claim 2, wherein the traditional Chinese medicine compound composition is prepared by mixing Semen Allii Tuberosi, Radix Morindae Officinalis, Epimedii Folium, Cistanche deserticola Ma, Lycium barbarum and Semen Cuscutae after separate extraction.

4. The application of claim 2, wherein the traditional Chinese medicine compound composition can be made into capsules, tablets or granules for improving male sexual function.
